(19) United States
(12) Patent Application Publication
(10) Pub. No.: US 2012/0116151 A1
(43) Pub. Date: May 10, 2012

(54) DEVICE FOR PREVENTING INCONTINENCE

(76) Inventor: Arne BRIEST, Karlsruhe (DE)

(21) Appl. No.: 13/261,097

(22) PCT Filed: Jun. 22, 2010
(86) PCT No.: PCT/EP2010/003757

§ 371 Date: Dec. 21, 2011

(51) Int. Cl. A61F 2/04

(52) U.S. Cl. 600/30

(57) ABSTRACT

The invention relates to a device for preventing incontinence, having a tube-shaped body (3; 129; 203) comprising a first longitudinal guide element (7; 143; 207) in the interior thereof. A hose-like retaining element connects to the tube-shaped body (3), comprising a second longitudinal guide element (13; 113; 213) in the interior thereof. Said second guide element (13; 113; 213) opens in the axial direction into a termination on the side thereof opposite the tube-shaped body (3). The hose-like retaining element is implemented as a reversible stretchable and collapsible metal mesh (23) comprising a polymer coating (25).

The invention relates to a device for preventing incontinence, or a part of this device in different variants, which can be combined with itself or with another variant to form the finished device.

The term incontinence basically refers to the inability to retain something. In the sense of the present invention this can for example refer to urinary incontinence. This type of involuntary loss of urine is a very commonly occurring illness, which can have various causes. The general designation urinary incontinence is, therefore, just the umbrella term for a whole range of very different types of disease, which can be distinguished and defined by their causes. Besides the incontinence due to stress or pressure, also known are motor urge incontinence and sensory urge incontinence. Again, to be distinguished from these as further causes of incontinence are obstructive overflow incontinence and functional overflow incontinence, supra-spinal and spinal reflex incontinence, and extra-urethral incontinence.

In relation to stress incontinence, a device for controlling the bladder functioning by the company Uromedica is known, which basically consists of two small, implantable balloons. These are implanted under the skin next to the bladder, in a short surgical operation. The expansion of the balloon is meant to guard against any involuntary micturition, by compressing the bladder in this way. The natural act of urination is not prevented by this method because the size of the balloon is selected so that for urination a normal bladder pressure is sufficient to allow the emptying of the bladder. It should still be possible for a doctor to adjust the fill quantity of the two balloons after the implantation.

A device of this type has become known from WO-A-98/56311. WO-A-98/56311 describes an expandable device for preventing urinary incontinence in which a tube-shaped guide or channel is connected to a balloon whose circumference can be changed, thereby making it adjustable. Here the tube-shaped guide penetrates the balloon axially in such a way that the guide extends into the balloon and passes through to extend out from it. The respective connecting areas between the guide and the balloon are closed by sealing so as to be liquid-tight (impervious to fluids), e.g. using silicone. Instead of sealing with silicone or a comparable chemical or polymeric adhesive, ultrasonic welding also known as an alternative for a possible sealing technology.

In the tube-shaped guide there runs in the longitudinal direction a first channel, which ends inside the balloon and opens into it. This channel is used to fill the balloon, for example with a liquid, as required, and thus cause it to expand, or to withdraw liquid and thus reduce the size of the balloon.

A second channel may be provided, which allows the device to be inserted in the region of the urethra of a human body. This channel accordingly terminates in an orifice at the end region of the guide extending above the balloon and into the body. This second channel has the other opening of its guide in the region between the balloon and its proximal end.

As suggested material for the balloon there are, firstly, chemical compounds which are themselves able to form a seal with the tube-shaped guide at the contact points. Examples of such compounds are stated to be crosslinked silicone gel, polyvinylpyrrolidone and gum karaya, among others. Besides the design already described, of the expandable device with a first channel through which the balloon can be filled or emptied, in WO-A-98/56311 it is also described how the balloon wall can be pierced by a coreless hollow needle and filled or emptied in this way. Other materials for the balloon that are named are a biocompatible non-springback elastomer, or an appropriate polymer mixture of polyurethane, polymers such as polyethylene, polytetrafluoroethylene (PTFE), polystyrene or polyether ether ketone (PEEK).

Moreover the wall of the balloon can be fitted with reinforcing structures. For this purpose the balloon is made with a double-wall, and the reinforcing structure is positioned between these walls. It can be made of fibres consisting of polyester, nylon, polypropylene, polytetrafluoroethylene (such as TEFLON) or other polymers characterised by a high degree of hardness or a high modulus of hardness. Here the fibres can form a mesh which is woven into a supporting structure that is positioned between the walls of the balloon. In one embodiment the fibres can be less elastic than the wall itself. Then the woven support structure has a loose weave so as to allow an expansion or contraction of the balloon wall.

Alternatively, the fibres of the reinforcing structure can also be basically non-elastic and woven, in which case the fibres are then knotted so as to again allow an expansion or contraction of the balloon wall.

The reinforcing structure also has the added function of retaining particles enclosed in the balloon that may be used instead of a liquid for expanding it, so that they cannot escape out into the region around the balloon. Here the fibres woven into the supporting structure can be fitted with a watertight “ripstop” function. In this way droplets, for example, that may form when the balloon is penetrated by the hollow needle for filling or emptying it, can also be caught and retained.

Regarding the particles which can be used instead of, on in combination with, a fluid to fill the balloon, WO-A-98/56311 presents various examples of particles whose size makes them suitable for first injecting into the balloon via a hollow needle, so that they then increase their own diameter only once they are inside, and thus cause the balloon to expand.

One example is particles that have a core with many arms extending out from it. These particles are passed through the hollow needle in the compressed state, and then expand after entering the balloon. Other examples of such particles can have a tube-shaped, elongated structure, which thus allows them to pass through the hollow needle. The elongated structure then also prevents the particles from flowing back through the hollow needle, or through the opening that is at first left in the balloon when the hollow needle is withdrawn.

Other embodiments of particles can be made of a hydrophilic material such as polyvinylpyrrolidone, polyethylene glycol, carboxymethyl cellulose or hyaluronic acid, which expands inside the balloon.

The journal “Der Urologe A”, publ. Springer Berlin, Heidelberg ISBN 0340-2592 (Print), 1433-0563 (online), Vol. 45, number 7, dated July 2006, described how the medical team of C. Kempkensteffen, S. Hinz, F. Christoph, S. Weikert, M. Schrader and M. Schostak of the Urological Clinic and University Outpatients Department, Charité, Berlin, have reported complications including balloon ruptures in connection with the device proposed by WO-A-98/56311.

Besides such a device, which is basically used for preventing urinary incontinence, from WO-A-2006/091786 there is also known a device and a method that basically relate to the anal region and therefore faecal incontinence. Here the elongated device has a chamber at one end in the longitudinal direction, with an opening that is fitted with a plug-like cap. This is made of a material that a needle, e.g. of a syringe, can penetrate and inject a fluid into the chamber through. The cap is surrounded by a self-closing outer wall, e.g. made of a silicone or polyurethane elastomer, which ensures that no leaky area is produced when the needle is inserted or removed. The chamber may also have a solid insert, e.g. of titanium, with a silicone envelope, which acts as a stop for the needle. An elongated, flexible, tube-shaped conduit is connected to the chamber, with a primary section that can exchange fluid with the chamber, and adjacent to this a second section into which a wire-like element can be inserted in order to move the device forward. At the other end of the tube-shaped conduit there is an expandable element, e.g. made of a silicone or polyurethane elastomer. This is adjusted to the desired size after implantation, using the injected fluid, and can be re-adjusted after the operation.

In another embodiment the expandable element can also basically be non-elastic, in which case it is expanded to a predetermined form. This is achieved by specially selecting the polymer materials that the expandable element can be made of. One example named in this connection is polyethylene terephthalate (PET).

Another device and method which basically relate to the anal region and thus faecal incontinence, are known from WO-A-2005/082276. In this device there are a number of balloon-shaped expandable elements, each consisting of a membrane made of a biocompatible material enclosing an interior space. This interior volume can be filled with a variable quantity of fluid as required, this being fed in via elongated, flexible, tube-shaped conduits in each case. These open into a filling region at their other end, which has several orifices for filling the cavity. They are then fitted with closing plugs that are self-sealing so that no leaky areas are left after filling via a needle.

Therefore starting from this prior art the present invention has the objective of preparing a device for preventing incontinence in which such a rupturing of the balloon is avoided, and where the device is designed so as to be generally lighter and more manageable and also with less side-effects on the organism.

This objective is solved according to the invention by a device for preventing incontinence which consists of two parts, and in which at least one part of the device has a tube-shaped body with a first longitudinal guide element inside it, with a hose-like retaining element that connects to the tube-shaped body, this being fitted with a second longitudinal guide element inside it that opens into a termination in the axial direction at the end opposite the tube-shaped body; and where the hose-like retaining element is designed as a reversible, extensible and collapsible metal mesh, which has a polymeric coating, thus keeping it airtight and liquid-tight both at the end cap and also in the region of the transition to the tube-shaped body.

It is specially preferred to have the hose-like retaining element filled with air under normal or atmospheric pressure. In this way many difficulties in preparing a device for preventing incontinence can be overcome in a straightforward manner, all of which presuppose a retaining element that can be filled with a fluid. The pressure that the inventive device, or the respective part of the inventive device, is intended to exert on the urethra to effectively prevent incontinence, depending on the anatomical particulars of the patient, is adjusted by stretching or collapsing the hose-like retaining element. This stretching or compressing has an effect rather like that of a spring.

The tube-shaped body is preferably designed as a tube-shaped, flexible hose made of at least one biologically compatible plastic or polymer, the material being selected from among polyurethanes (PU), polyether block amides (PEBA), polyamides (PA), latex, polyvinyl chloride (PVC) and/or silicone.

One should specially mention the polyether block amides (PEBA) which are commercially available under the trade name PEBAX®. Compared to other thermoplastic elastomers such as the polyurethanes, these thermoplastic elastomers have the virtue of having a lower density. Moreover they have excellent mechanical and dynamic properties. Thus they have an excellent elasticity, impact resistance and endurance strength.

The hose preferably, but not necessarily, has in addition a hydrophilic coating. This hydrophilic coating increases the lubrication of the hose surface, which firstly facilitates the implantation, and secondly contributes to an increase in the comfort of wearing the implanted device.

Other preferred options are fabric-reinforced and/or fabric-woven flexible hoses for securing an improved transmission of force. Here one can mention for example silicone hoses with monofilament polyester fabric inserts. Alternatively a peroxide-linked, monofilament polyester fabric may be used. A different synthetic material such as PEBA would also suitable instead of polyester. The fabric insert can be a single or multiple fabric insert.

These hoses can also be fitted with a metallic reinforcement. Thus they can be reinforced by a metal mesh, where because of the special hygiene requirements platinum should be specially mentioned as a suitable metal. A metal reinforcement in the form of a metal spiral may be provided.

Alternatively the tube-shaped body can be designed as a so-called shrink-hose made of at least one biologically compatible thermoplastic. Suitable thermoplastics for this purpose are mainly polyolefin, polyvinyl chloride (PVC), polyvinylidene fluoride (PVDF), polytetrafluorethylene (PTFE) and/or Viton. Here Viton® is the name of a plastic that is commercially available from the company DuPont. If such a shrink-hose is used, the tube-shaped body and the hose-like retaining element with the metal mesh can fit together almost seamlessly. No special connection, such as by a marker band or a clamp connector, has to be provided.

If the tube-shaped body made of the shrink-hose material is additionally formed as a fabric hose with a metal insert in the form of e.g. a platinum mesh, by the so-called “braiding” method of manufacture, then the fine metal wires can extend into the hose-like retaining element and so end inside the hose-like retaining element. What is more, by extending in this way the metal mesh of the hose-like retaining element is reinforced advantageously in the region of the transition from the tube-shaped body to the hose-like retaining element. Shrink-hoses are available in the trade in many different designs. One can choose either thin-walled, medium or thick-walled hoses.

If the tube-shaped body is designed as a fabric-reinforced and/or fabric-woven flexible hose and has a metal reinforcement, then it is also possible to design the metal reinforcement and the reversible, extendable and collapsible metal mesh of the hose-like retaining element in the form of a sandwich between two plastic layers. Here the metallic reinforcement and the metal mesh can interweave.

In one specially preferred embodiment the tube-shaped body has an anti-microbial silver coating or a coating made of a diamond-like carbon or DLC. These coatings are extremely thin and they each serve to minimise the settling of germs.

In another embodiment of the inventive (partial-) device, attention is paid to the fact that the demands on the properties of the outward-facing surface of the device, and the demands on the precise positioning after the implantation, may basically be opposed to each other. While the said surface needs to be as smooth as possible, to avoid irritation of the surrounding tissue, on the other hand a good anchorage in the tissue is required in order to prevent any slippage of the device. This problem can basically be countered by a topological surface modification, which the invention provides, in that the tube-shaped body has a basically closed surface envelope, on which outward-facing fastening elements in the form of protrusions are moulded on the tube-shaped body and designed to be of one piece with it.

These protrusions may be moulded on the tube-shaped body at regular intervals, preferably in rows, or at irregular intervals.

These are preferably moulded on the tube-shaped body in the form of knobs, hooks and/or as scale-like protrusions.

These measures make it possible for the device to stay in place with a precise fit after it has once been implanted, especially if it is to be re-adjusted from the outside at a later date.

The metal mesh of the hose-like retaining element can be formed as a diamond pattern, where the rhombuses assume the form of slits when in the compressed state, extending in the lengthwise direction of the flexible hose. It is specially preferred that the slits, and thus the rhombuses of the metal mesh, have different sizes. In this way the stress loading on the mesh material, for the re-adjustable and the actively adjustable embodiment, is considerably reduced, and so the life of the material is optimised for long-term use.

If the part of the inventive device is designed so as to be adjustable, then the tube-shaped body has, as a first longitudinal guide element, a flexible shaft approximately in the middle, which is held in the tube-shaped body by spacers.

According to one embodiment of this adjustable part of the device, the flexible shaft opens at its distal end section into a control element, which in turn has a slot at its distal end that can be used for manual adjustment, e.g. with a screwdriver, or for adjusting via the connection to a motor as drive unit. In the case of motor-driven adjustment the control element is preferably a spindle.

If the inventive device according to another embodiment is not re-adjustable, the tube-shaped body of at least one part of the device is designed as a tube the outside of which is at least partially surrounded by a sleeve. The hose-like retaining element opens into an insert which receives the second longitudinal guide element in the form of a threaded rod, and the pin acts as a stop for the longitudinal guide element and the insert.

Here the tube, the sleeve and the pin can each have coupling elements which match up with an identical or complementarily formed coupler element in the insert in the hose-like retaining element. In this manner a secure and yet relatively simple connection between the longitudinal element and the retaining element of the inventive device are produced.

According to another embodiment at least one part of the inventive device is designed so as to be actively, freely adjustable and re-adjustable. Here the tube-shaped body has as a first longitudinal guide element a toothed rod approximately in the middle, which is held in the tube-shaped body by spacers, and which can be moved via a lifting device mechanically by hand or motor-driven. This lifting device is preferably a piston.

This part of the actively adjustable device can be coupled to a sensor detecting motion, inclination, pressure and/or volume, and controlled by at least one of these sensors.

The types of sensor that can be used according to the invention make it possible to have access to the basic external and internal factors which could have an influence on the proper use or proper working condition of the device for preventing incontinence, for purposes of inspection and control. Here both the external parameters, such as the barometric pressure, and also internal parameters, i.e. those that are physiology-based, can be measured, for example using the volumetric sensor.

All the said types of sensor are already known in the field of medicine and are being used for various applications. It is advantageous for them to be fitted externally and thus avoid encumbering the implantation site unnecessarily. Because the air column formed inside the hose-like retaining element it at atmospheric pressure, it is subject to the usual fluctuations of air pressure. More extreme conditions, such as air-pressure fluctuations when flying, or even at altitudes above at least 2000 m, may certainly cause a change. This is taken care of by monitoring the inventive device or the particular part of the device. For this purpose the aforementioned sensors collectively, or alternatively at least one or two of them chosen for the specific purpose, can be deployed.

The embodiments of the inventive device described above, including the mentioned variants, each apply to one part of the device for preventing incontinence. The finished device has two identical or different parts from among the parts of the device that have been presented here. Each of these are to be implanted adjacent to the urethra of the patient.

Each of the described embodiments of the inventive (part-) devices can now be implanted in a human body in such a way that they are combined as a pair or combined with one of the other parts of the device that have already been described previously.

It is thus possible to assemble the finished device for preventing incontinence in very different ways. One option is to have two parts of the re-adjustable and/or actively re-adjustable device, as described in detail above, implanted next to the urethra of a patient and so form the final device.

Another option is to have one part of the device re-adjustable and/or actively re-adjustable, while the other part of the device is selected as a non-re-adjustable variant, and both of the different parts are implanted adjacent to the urethra of a patient so as to form the final device.

It is of course also possible for the finished device to have two parts of the non-readjustable variant of the inventive device.

In what follows the invention will be described in greater detail using exemplary embodiments and the enclosed drawing.

It shows:

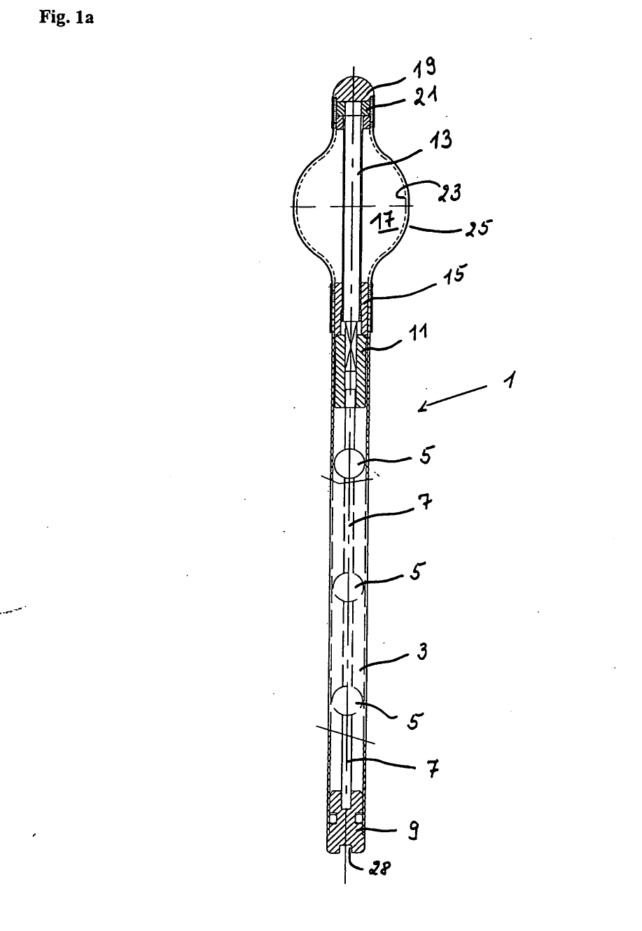

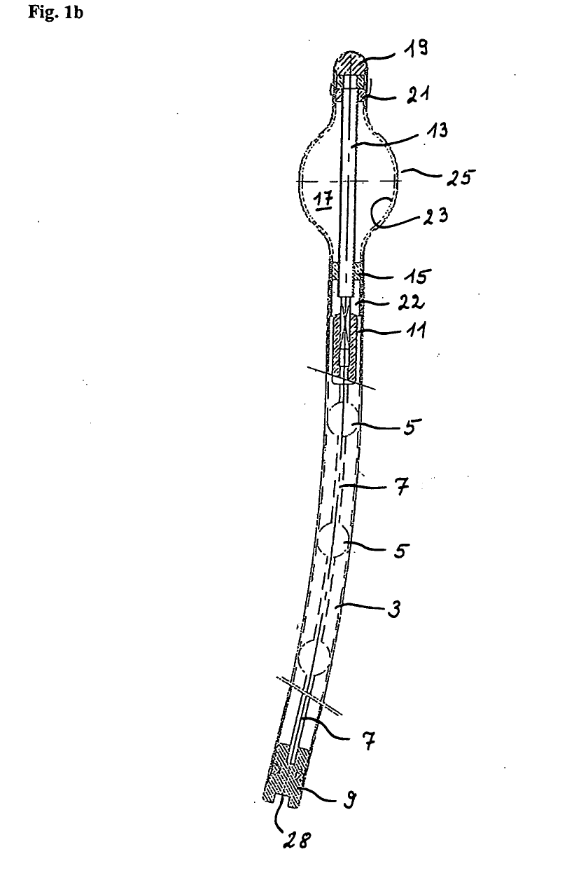

FIG. 1a a schematic, sectional view of a device for preventing incontinence in a first, adjustable embodiment,

FIG. 1b a schematic, sectional view of a device for preventing incontinence as depicted in FIG. 1a with slight re-adjustment,

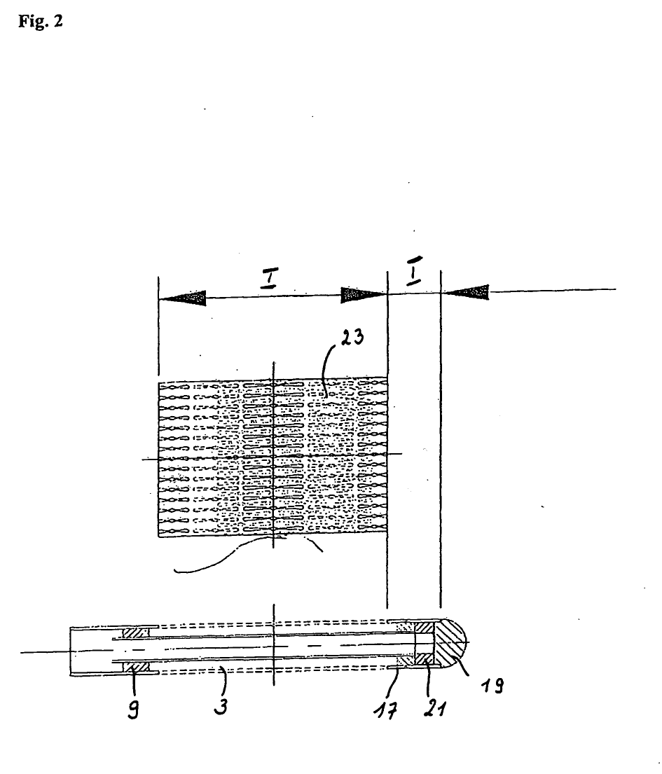

FIG. 2 a schematic, sectional view of the metal mesh in un-expanded form as detail view, from the section I of the complete device of the first exemplary embodiment, shown next to it,

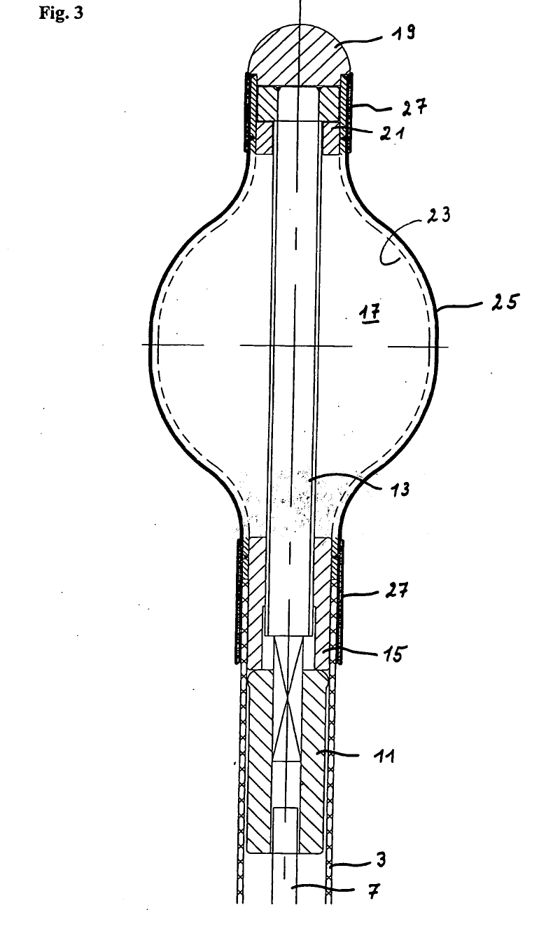

FIG. 3 a schematic, sectional detail view of the inventive device according to FIG. 1a, which shows the region where the flexible hose is connected to the cage,

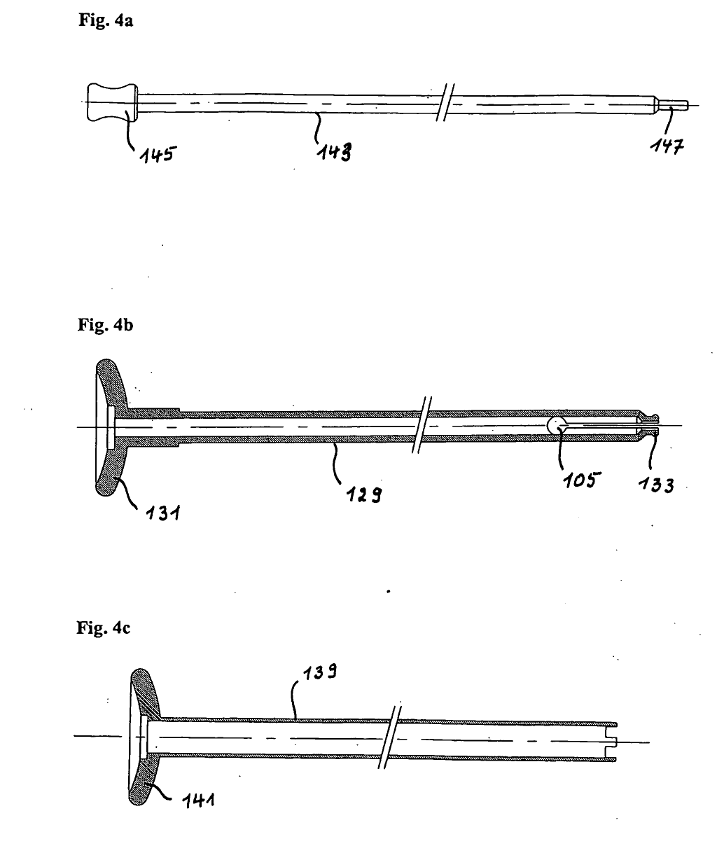

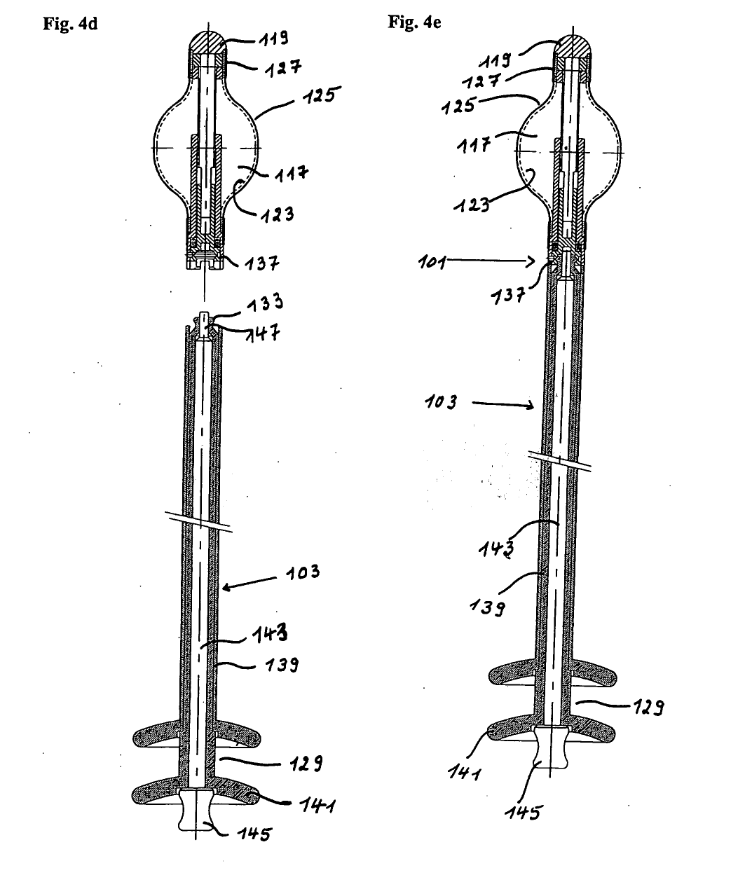

FIG. 4a a schematic, sectional view of the pin as part of the longitudinal guide element of the inventive device in a second, non-adjustable embodiment,

FIG. 4b a schematic, sectional view of the tube as part of the longitudinal guide element of the inventive device in a second, non-adjustable embodiment,

FIG. 4c a schematic, sectional view of the sleeve as part of the longitudinal guide element of the inventive device in a second, non-adjustable embodiment,

FIG. 4d a schematic, sectional detail view of the inventive device in a second, non-adjustable embodiment, before the coupling of the longitudinal guide element to the cage,

FIG. 4e a schematic, sectional detail view of the inventive device in a second, non-adjustable embodiment, after the coupling of the longitudinal guide element to the cage,

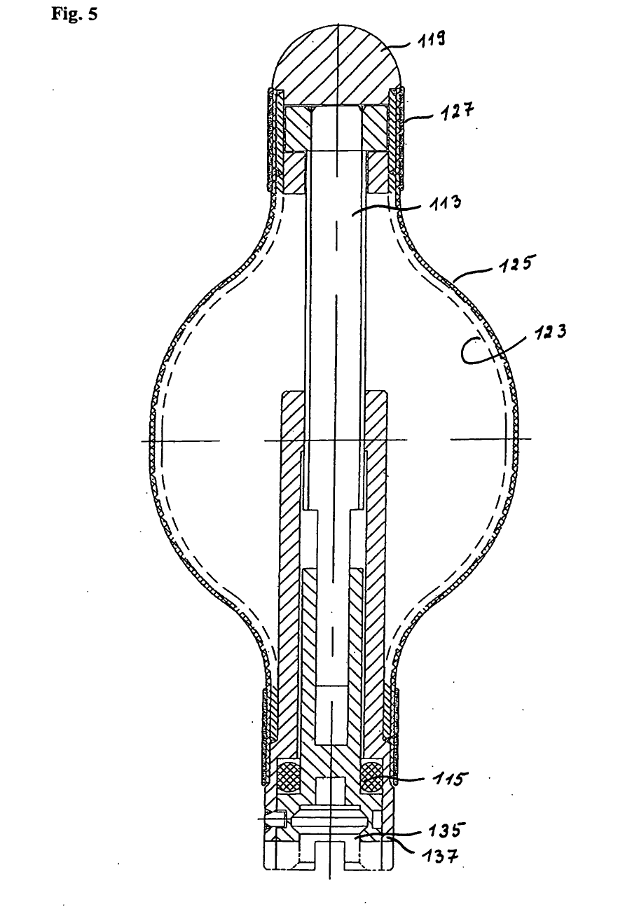

FIG. 5 a schematic, sectional detail view of the inventive device according to FIG. 4d, which shows the region of the connection of the longitudinal guide element to the cage,

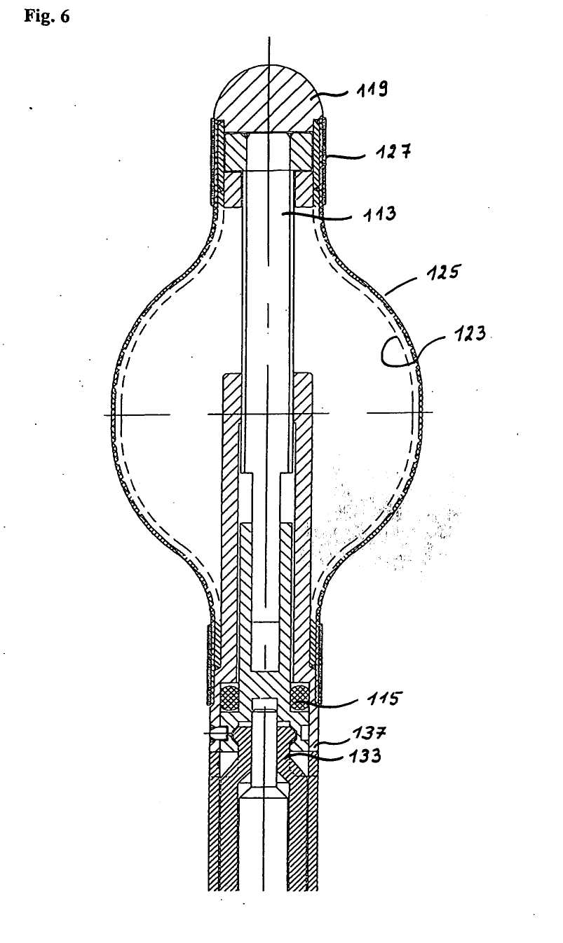

FIG. 6 a schematic, sectional detail view of the inventive device according to FIG. 4e, which shows the connection of the longitudinal guide element to the cage,

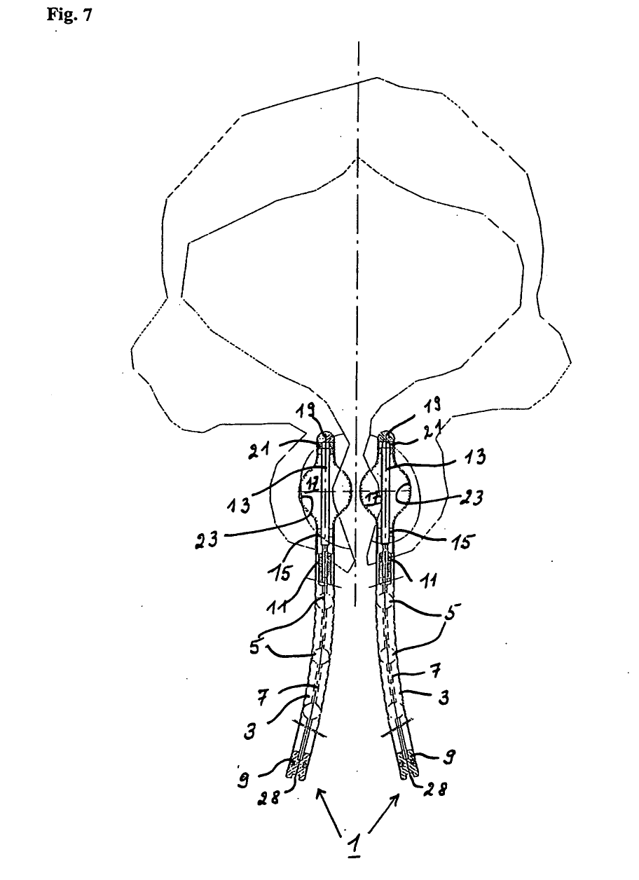

FIG. 7 a schematic, sectional view of two adjustable inventive (part-) devices after being transplanted into a lumen in the body,

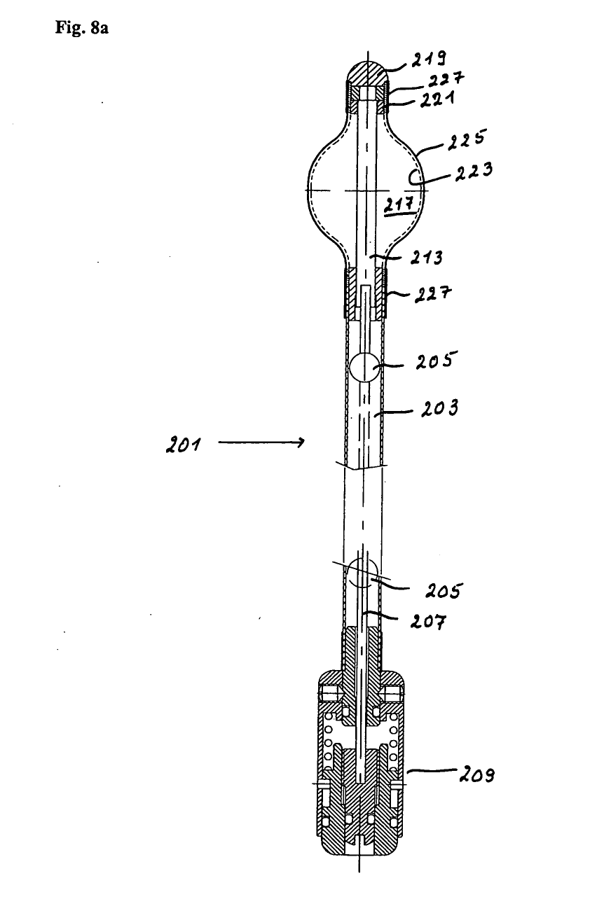

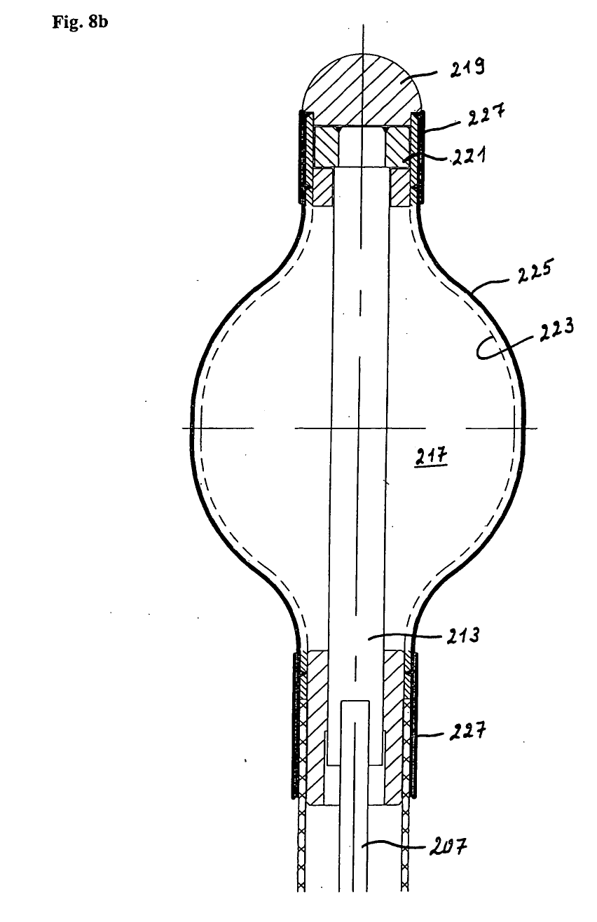

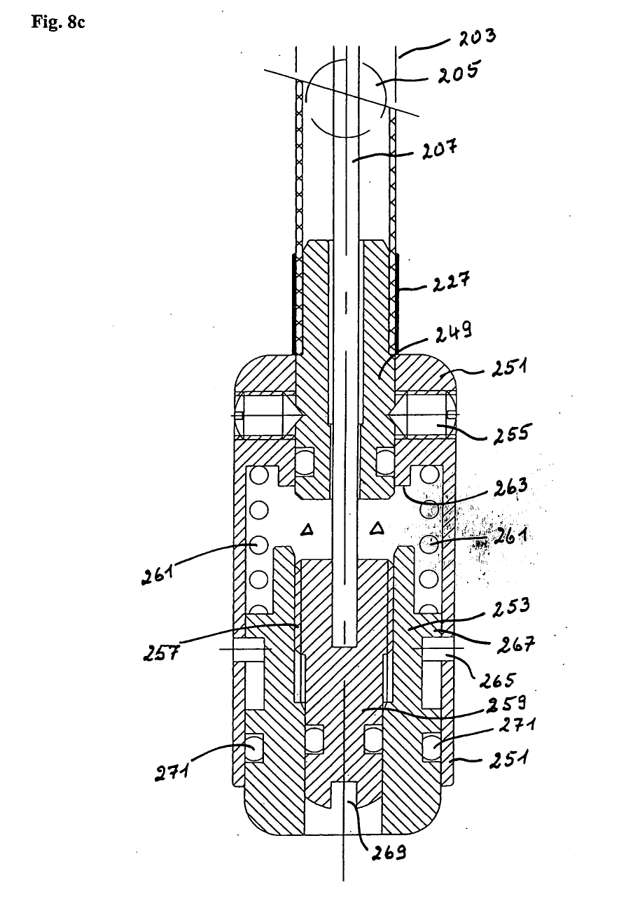

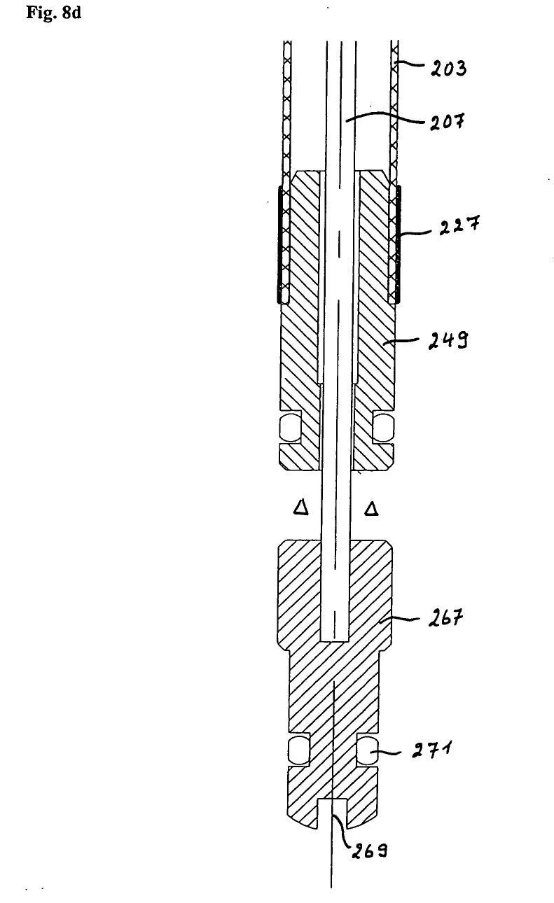

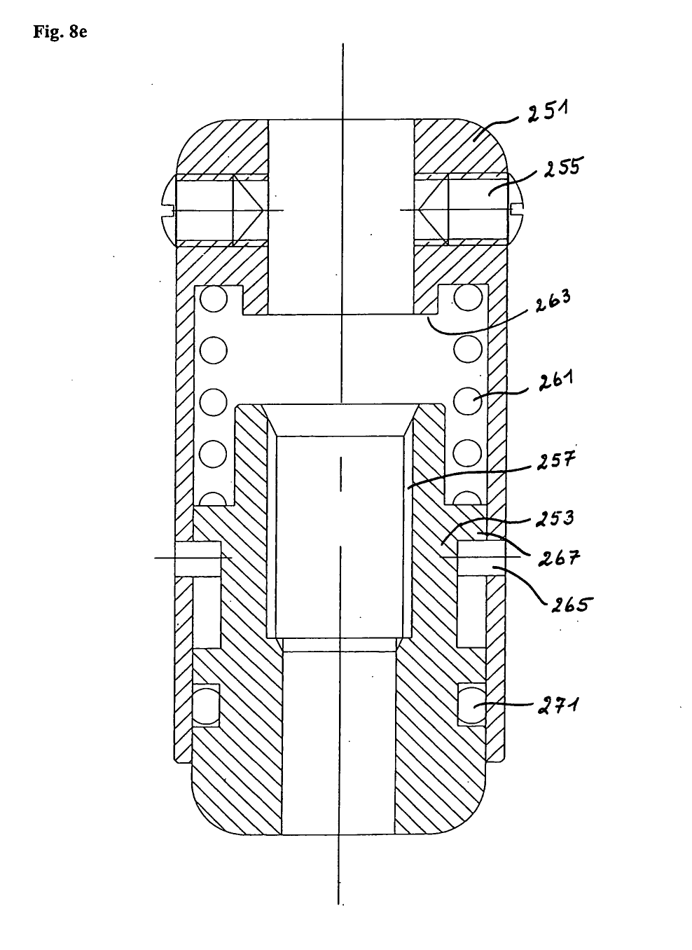

FIG. 8a a schematic, sectional view of a device for preventing incontinence in a third, actively adjustable embodiment,

FIG. 8b a schematic, sectional detail view of the inventive device according to FIG. 8a, which shows the connection of the flexible hose to the junction to the cage,

FIG. 8c a schematic, sectional detail view of the inventive device according to FIG. 8a, which shows the control element as adjusting unit,

FIG. 8d a schematic, sectional detail view of the inventive device according to FIG. 8a, which shows the guide and the support of the toothed rod in greater detail, and

FIG. 8e a schematic, sectional detail view of the inventive device according to FIG. 8a, which shows in greater detail the outer sleeve with the piston of the control element that it contains, as adjusting unit,

### 1ST EXEMPLARY EMBODIMENT

### Adjustable Device

FIG. 1 a shows an inventive device for preventing incontinence, or a part of such a device 1, designated as a whole by the reference 1. The device 1 is made up of two parts, because the requirement is that it works together with another part of the device 1 that operates in the same way, or in a different way which is described below, in order to achieve the desired success, this again being described in more detail below. Firstly the device 1, which thus represents just one part of the device 1 in its final form ready for use or operation, will be described in more detail by referring to FIG. 1a. This part which is to be explained below will nonetheless still be referred to as device 1, for the sake of simplicity.

The device 1 has a tube-shaped, flexible hose 3, which is made of a biologically compatible plastic or generic polymer, so that it can be implanted in a human body. In the course of testing, during preliminary tests a polyether block amide (PEBA), commercially available under the trade name PEBAX®, and also latex, polyvinyl chloride (PVC) and silicone; have been found to be equally suitable. It should be obvious to an expert here that by citing these plastics or polymers, suitable materials have been stated by way of example, without intending this to be restrictive. With regard to latex one should mention that besides natural rubber, synthetic rubbers are also suitable.

In particular during the preliminary tests a silicone hose was used bearing a monofilament polyester fabric insert. In a second, slightly modified preliminary test, the same hose was used but with a platinum mesh construction. It was possible in this way to achieve a hardness of approx. 70° Shore A.

Yet another preliminary test was done using a peroxide-cross-linked, monofilament polyester fabric as reinforcement of the silicone hose.

The reinforcement by the fabric insert and the platinum mesh serve the purpose of enabling an optimised transfer of force.

All the tests were concluded with very good results as regards the durability and toughness of the hoses used.

To ensure that it keeps its shape, spacers 5 are fitted in the flexible hose 3, being held by a flexible shaft 7, which passes through approximately the greater part of the length of the flexible hose 3. In the present exemplary embodiment this flexible shaft 7 is made of a nickel-titanium alloy, which has superelastic properties and a low modulus of elasticity. The important feature of this type of alloy is its form-retaining, memory effect. This is made use of to give the shaft its shape at body temperature, by applying a force to adjust it appropriately.

This flexible shaft 7 opens at the distal end, i.e. the end facing out from the respective centre of the body in a planned implantation, of the flexible shaft 3 in an control element 9, the function of which will only be explained in more detail below. At its proximal end i.e. facing towards the body, it opens out to a coupler 11. Here this is an elastic coupler 11 made of an elastomer. The coupler 11 is elastically bendable. It serves as connecting element to a threaded rod 13, which is held by a threaded nut 15 at the proximal end of the flexible hose 3. Approximately in this region there is also the transition from the flexible hose to a cage, denoted as a whole by 17, which is shaped in such a way that by a combined action with the flexible hose 3 it can effectively prevent incontinence in a patient. The threaded rod 13 passes through the cage 17, which holds air under normal pressure, to open onto a closing plug 19, which closes off the cage 17 towards the outside so as to be airtight and fluid-tight, and at the same time serves as axial bearing 21 for the threaded rod.

The mode of operation of the inventive device is best made clear by a comparison between the embodiment shown in FIG. 1a and the depiction in FIG. 1b. Here it is basically one and the same part of the device, no different variant is shown in FIG. 1b. It is only to show the effect of a force that acts on the control element 9 in a manner which is yet to be described, which moves the flexible shaft 7 and thereby draws out the cage 17. By this the cage 17 takes on a more elongated form than that shown in FIG. 1a. In the implanted state this means that the pressure on the urethra, in the immediate vicinity of which the implant is positioned in the body, is reduced. The effect exploited here can be roughly compared to a the working of a spring.

This demonstrates a fundamental difference between the mode of operation in the prior art for adjusting the support element which is there formed as a balloon, and the adjustment of the inventive cage. While the balloon according to prior art is basically filled with a fluid and is, for example, filled up with yet more fluid or some comparable medium via a hollow needle, in order to expand it, or the fluid is withdrawn from it by means of the hollow needle should a narrowing or reduction in size be required, in this invention the adjustment is done by changing an air column. The form of this air column is changed by a force acting on the control element 9, which acts on the shaft 7, moving this together with the coupler 11, and thereby pulling the cage 17—in the example in FIG. 1b—to stretch it more lengthwise. A cavity or room for play 22 is formed between the coupler 11 and the threaded rod 15.

The cage 17 is made of a fine metal mesh 23, shown in FIG. 2, such as has recently become known e.g. from the field of carotid surgery for catheter operations using metal-mesh stents, where it is also used. Here the fine metal mesh 23 is designed with a diamond-pattern, because it needs to have the capacity for extending as required when the cage 17 expands. This means that by designing it to have a diamond-shape, the metal which is not actually extensible as such, but still does provide a very durable material, can be utilized because it makes possible an expansion—when the rhombuses are stretched—and a contraction—where the diagonal distances across the rhombuses are reduced. In this manner the material metal is rendered flexible.

In FIG. 2 a sample section of the cage 17 in its non-expanded state is shown, and the complete device 1 in non-expanded form is again shown next to it. One can see from this picture that the diamond-pattern grid structure is pulled together to form slits that extend in the longitudinal direction of the flexible hose 3. Tests have shown that the stress acting on the material can be further optimised if the slits, and thus the diamond-shapes of the metal mesh 23, are of different sizes.

The metal mesh 23 of the cage 17 is additionally covered by a plastic layer making it airtight and fluid-tight. In the exemplary embodiment this is achieved by drawing a PTFE sheath 25, visible on FIG. 3, over the metal mesh 23, and holding it in place by a marker band or a clamp connector 27 which is shown in detail in FIG. 3. The sealing against fluids can, however, also be achieved by means of a silicone coating or by coating the metal cage 17 with a comparable plastic.

The inventive device for preventing incontinence 1, as shown in FIG. 1a and FIG. 1b, and described in more detail here, represents an adjustable inventive device 1. Here the re-adjusting can be done in various different ways. In the simplest case the slot 28 forms the grip for a screwdriver, using which the flexible shaft 7 with the coupler 11 is moved forward or backward, thus stretching or compressing the cage 17 in this simple way.

In another embodiment the part of the device 1 described here is fitted with a motor drive. The motor itself is not shown in FIG. 1. Here the control element 9, as a spindle, forms the connection between the motor and the device 1. The spindle is then mounted on a ball bearing, which in the exemplary embodiment is designed as a hybrid ball bearing, but could just as well be an ordinary inclined ball bearing.

FIG. 3 again shows a detail view of the adjustable embodiment of the inventive device 1 which is being described in detail here, with coupler 11, threaded nut 15, and the adjoining cage 17 with its airtight and fluid-tight coating 25. The clamp connector 27 positioned both at the distal and proximal ends of the cage 17 not only fastens the PTFE sleeve 25—or any other desired plastic sleeve—on the cage 17, but also connects the cage 17 to the PTFE sleeve 25 in an airtight and fluid-tight manner to the flexible hose 3, and accordingly seals the cage 23 with the PTFE sleeve 25 opposite the closing stopper 19 in the proximal end region of the cage 23 so as to be airtight and fluid-tight.

### 2ND EXEMPLARY EMBODIMENT

### Non-Adjustable Device

This other, second embodiment of the inventive device 101 differs from the first embodiment basically in that it is not re-adjustable later on, i.e. after the filling and implantation have been carried out. In this case the components that are the same as in the first embodiment are labelled with the same reference numbers, but added to 100.

Here the longitudinal guide element, designated as a whole by the reference 103, instead of the flexible hose is constructed like a trocar but nested in three interlocking parts, as shown on FIG. 4a-4c. A tube 129, after the filling has been done, forms a part of the guide element 103 positioned in the middle of this nested structure. Because in the case of this embodiment no re-adjustment is intended, the tube 129 is missing either a flexible shaft 7 inside it or the control element 9 which was provided at the distal end region in the first embodiment. The tube 129 has a wing-shaped extension 131 at its distal end region, running round its entire circumference, while at its proximal end region it tapers to form an appendage 133. The appendage 133 is aligned so as to form a precise fit with a recess 135 which is shaped to match it, and which is located at the distal end region of the cage 117, and is shown in a detail view in FIG. 5. In this manner the appendage 133 and the recess 135 form a coupler to make up the finished device 101, or at least this one part of the device.

The recess 135 is formed in an insert 137 which serves as bearing and connecting element for the threaded rod 113, and also to guide it. The threaded rod 113 is held at the distal end of the cage 117 by means of a threaded nut 115, which forms a part of the insert 137. In the case of this embodiment the transition from the tube 129 to the cage 117 also takes place in this region. The threaded rod 113 passes through the cage 117, which is designed in the way described above in the first embodiment, so as to open into the closing plug 119, which closes off the cage 117 towards the outside making it fluid-tight, and at the same time serves as axial bearing 121 for the threaded rod 113.

A sleeve 139 which serves as reinforcement, shown in detail in FIG. 4c, is pushed over the tube 129. The sleeve 139 has a wing-shaped extension 141 at its distal end region, running round its entire circumference, while at its proximal end region, with the diameter remaining the same, it meets the insert 137, which in turn meets the taper of the cage 117 provided at the distal end region of the cage, forming an exact fit. In this manner the sleeve 139 is coupled to the insert 137 in an exact fit.

As final element there is also a pin 143, which is inserted into the tube 129 after the sleeve 139 has been put on the latter. This pin 143 has a bulge 145 at its distal end and a taper 147 at its proximal end. The length of the pin 143 is chosen to match the length of the tube 129 in such a way that the bulge 145 rests outside on the distal, wing-shaped extension 131 of the tube 129, just as the slightly inclined end section before the taper 147 at the proximal end of the pin 143 meets to form an exact fit inside the matching slight taper of the appendage 133 at the proximal end of the tube, and the taper 147 of the pin 143 itself reaches through the appendage 133 of the tube 129. Here the taper 147 of the pin 143 protrudes slightly from the appendage 133 of the tube 129, just enough to fit exactly inside the recess 135 of the insert 137. In this way the pin 143 forms the connection between the longitudinal element 103 and the cage 117, and acts as a key locking the two together, thus holding the inventive device 101 according to this embodiment.

FIG. 4d shows in more detail how the three parts of the longitudinal element 103, in the form of the tube 129, the sleeve 139 pushed over it, and the pin 143 inserted inside the tube 129, fit inside each other. Here the taper 147 of the pin 143 protrudes from the proximal end of the tube 129. The adjoining cage 117, shown as still separated at a distance from the longitudinal element 103, with its fine metal mesh 123, which here is again designed as a diamond-pattern, is fitted with the PTFE sheath 125, and the clamp connector 127 that has already been explained above for the first embodiment. This is found both at the distal and proximal end regions of the cage 123. It not only fastens the PTFE sheath 125—or any other desired plastic sleeve—to the metal mesh 123 of the cage 117, but also similarly connects the cage 117 with its PTFE sheath 125 in a fluid-tight way to the insert 137, which at the same time serves as a mount for the threaded rod and as a coupler component for the longitudinal element 103. The clamp connector 127 likewise forms a fluid-tight seal between the cage 123 with its PTFE sheath 125, and the closing plug 119 in the proximal region of the cage 123.

FIG. 4e shows the coupling of the longitudinal element 103 to the cage 117, and how the taper 147 on the pin 143 thus fits exactly inside the insert 137.

FIG. 6 again shows a detail view of the cage 117, with the insert 137 held by the clamp connector 127, which both holds the threaded rod 113 via the threaded nut 115 and also bears the taper 147 of the pin 143 inserted inside it.

### 3RD EXEMPLARY EMBODIMENT

### Device with Fastening Elements

In what follows a modified embodiment of the inventive device 1 for the prevention of incontinence, or of the part of the device and the way it works together with a second part, as the case may be, will be described. Here the structure of this device 1, as described above, stays basically the same. The modification pertains to measures to avoid slippage of the device 1, effectively and while being as gentle on the organism as possible. These are shown by way of example on the device described for the 1st embodiment, and as such are not shown in more detail on the FIG.

Hence in the descriptions that follow the reference numbers for the 1st embodiment are used.

According to this modification the tube-shaped, flexible hose 3 is fitted with fastening elements in the form of protrusions distributed around its circumference on its sheath surface which is closed to the outside, and which may be arranged in a regular or irregular pattern.

These are basically moulded in one piece with the sheath surface of the hose, so as to be elastic or elastically springy. Here they mainly point radially out from the hose 3, which means that they are constructed at an angle of approximately 90° to the longitudinal direction of the hose 3. In this manner they block all movements, including fine movements, of the hose 3, that might otherwise result in it drifting away from its implantation site.

In the embodiments tested as examples, the protrusions were formed as rows of knobs arranged in both regular and irregular patterns.

In fact another design was also tested, which is again not shown here, in which the protrusions were shaped as scales. This produced a general ribbed type of surface structure, which has as a whole proved to be very gentle on the surrounding tissue.

The materials that have already been suggested above for the first embodiment are suitable materials for the fastening elements, because of the design where they are one of a piece with the hose 3. Thus they are already moulded at the time when it is manufactured.

### 4TH EXEMPLARY EMBODIMENT

### Another Device with Fastening Elements

Another modified embodiment of the inventive device 1 for the prevention of incontinence, again relates to measures which are intended to prevent any slippage of the device 1, effectively and as gently as possible. Here for the purpose of explanation, the construction of the adjustable device is taken as starting point, as described in detail for the first exemplary embodiment, and again a material that has a memory-effect is chosen for the flexible shaft 7. Here it has proven effective if the flexible shaft 7 is made of a nickel-titanium alloy as in the 1st exemplary embodiment. And here nitinol has proved especially effective, made with a nickel component of about 50%, and which is pseudoelastically deformable by up to about 8%.

The flexible shaft 7 is now basically designed to be built up in sections, where hollow pipe-shaped sections alternate with rod-shaped distancer pieces each of about the same length. These are then at first found in a start position referred to as the “elongated state”.

The special feature of the pipe-shaped sections is their structure, which first becomes visible when the flexible shaft 7 is changed to its outward-bulging shape. Thus the pipe-shaped sections are now shifted with respect to each other so that the distance between them is reduced as the distancer pieces are pushed inside the tube-shaped sections. This is possible thanks to the structure of the pipe-shaped sections which now expand outwards in the form of stud-like, rounded ridges which are gentle on the tissue. Along the flexible shaft 7 there is formed a surface structure with bulges rather like knobs, that ensures a firm seating of the device at the implantation site.

Alternatively several hollow, pipe-shaped sections may be arranged in a row together before the next distancer piece comes, provided this does not impair the stability of the flexible shaft 7.

In the case of these forms of the 4th exemplary embodiment the distancers 5, as shown for example in FIG. 1a, are no longer required.

### Implantation of the Inventive Device in a Body Lumen According to a First Variant

FIG. 7 shows the implantation of the inventive adjustable device 1 for preventing incontinence inside a body lumen. Here in each case two of the devices 1 are implanted adjacent to each other so that the urethra of a patient afflicted with urinal incontinence is held in the middle between them. The cage 17 in the design described above for the first embodiment is filled with air. Only a minimally invasive operation is required for implanting the two parts of the device 1.

If for example the two devices are used for a male patient following a prostatectomy, they are implanted in the immediate area of the operated prostate.

If for example the two devices are used for a female patient following a hysterectomy, they are implanted in the immediate area of the bladder.

The expansion of the cage 17 is chosen precisely so that when the two devices 17 are in their final set, i.e. adjusted, position, a completely normal urination with a normal bladder pressure is not impaired, nor is it partially or wholly prevented. The air columns in the two cages 17, and their position next to the urethra, are adjusted by means of the flexible shafts 7 that are each connected proximally to the coupler 11, so as to ensure that only an involuntary urination, for example in the case of stress incontinence, is prevented. Such incidents may happen if the patient is compelled to cough, sneeze or make other such sudden movements. The stress incontinence may also be triggered by lifting of loads.

The precise adjustment of the cage 17 is done by two different setting procedures, either manually or motor-driven.

For manual operation the slot 28—as already indicated above—provides the grip for a screwdriver. In model tests, as an alternative the mounting of an elongated screw was tested, which is not shown in detail on FIG. 7, which extends to fit precisely into the slot 28, and which can then in turn be operated using the screwdriver. Both variants were found in the model tests to be practicable.

In the case of motor-driven operation, a motor is connected via the control element 9 to the device 17, and it is used to move the flexible shaft 7 with the coupler 11 forward or backward, thus stretching or collapsing the cage 17 in this simple way. The flexible shaft 7 is then designed as a shaft in the form of a motor spindle that is directly driven, and mounted via the control element 9.

For precise fine adjustment, magnetic valves moved by an electromagnet, cantilevers, or piezo adjusting elements have been found to be equally suitable, the latter type being specially sensitive and allowing a specially good fine adjustment.

To adjust the two parts of the inventive device 1, one first ensures that the parts are inserted by a minimally invasive operation, and then the wound is initially left open for at least one day, a procedure that is quite straightforward in minimally invasive surgery. If an elongated screw is used, which extends to precisely fit into the slot 28, this is also implanted during the operation. The same holds for the connection of a motor via the control element 9, as described above.

After the two parts of the device 1 have been properly adjusted, the elongated screw or the motor connection can be removed and the wound closed.

### Implantation of the Inventive Device in a Body Lumen According to a Second Variant

This second variant for the implantation of the inventive device differs from the aforementioned first variant in that here the first exemplary embodiment, which relates to the adjustable device, is combined with the second exemplary embodiment, which features the non-adjustable design. Thus an adjustable device is implanted in the region of the bladder on one side, and on the other, opposite side, a non-adjustable device is implanted. This variant, too, allows an adjustment to be made taking into account the actual pressure on the urethra required at the location. Model tests have shown that this adjustment on just the one side can suffice for a successful functioning of the device.

In this manner a variant that is less expensive and easier to manufacture is produced, without having to forego the advantage of adjustability.

### 5TH EXEMPLARY EMBODIMENT

### Actively operable, adjustable device

FIG. 8a-8e show another design of the inventive device for preventing incontinence, which is actively adjustable even after the implantation and remains so. In this case, again the components that are the same as in the first embodiment are labelled with the same reference numbers, but added to 200 here. Accordingly the inventive device as a whole is labelled with the reference number 201.

As can be seen from FIG. 8a, the device 201 has a tube-shaped, flexible hose 203, which is again made of a biologically compatible plastic or generic polymer, so that it can be implanted in a human body. For this one his referred to the first exemplary embodiment.

To ensure that it keeps its shape, spacers 205 are fitted in the flexible hose 3, while the inside rod is now not formed as a shaft but as the toothed rod 207, which passes through the whole length of the flexible hose 203. The toothed rod 207 opens at the distal end of the flexible hose 203, that is to say the end facing away from the middle of the respective body when a planned implantation is being carried out, into the control element 209, which according to this variant of the inventive device is constructed as an adjusting unit, which the patient, in whose urethra region the device is implanted, or the doctor who is looking after the patient, can operate to re-adjust the device.

As FIG. 8b shows in detail, the toothed rod 207 opens at its proximal end, i.e. at the end nearer the body, into a junction 211 in a threaded rod 213, which runs through the cage 217 and is held at the proximal end of the flexible hose 203 by means of a threaded nut 215. Approximately in this region there is also the junction between the flexible hose 203 and the cage 217, which has already been described in detail in the first exemplary embodiment, so here we will simply refer to that description. The closing plug 219 closes off the cage 217 towards the outside so as to be airtight and fluid-tight, and at the same time serves as axial bearing 221 for the threaded rod.

For the plastic which also envelops the metal mesh 223 of the cage 217 so as to be airtight and fluid-tight, which is described in detail in the first exemplary embodiment, in this exemplary embodiment a PTFE sheath 225 is used, which is pulled over the metal mesh 223 and held by a marker band or a clamp connector 227 fastened at both sides to the sheath. The sealing against air or liquid can, however, also be achieved by means of a silicone coating or by coating the metal cage 217 with a comparable plastic.

In what follows the control element 209 of the inventive device for preventing incontinence 201, which is formed as an adjusting unit, will be described in more detail, as depicted in detail particularly in FIG. 8-8e.

The control element 209, designed as an actively adjusting unit, is held with its proximal end inside the flexible hose of the longitudinal guide element 203, at the distal end of the latter. For this there is a marker band or a clamp connector 227 fastened on the outer circumference of the hose. The said proximal end of the control element 209 forms a guide 249, which receives the distal end, i.e. the end facing away from the body, of the toothed rod 207, and thus serves as its mount. Furthermore the control element 209 according to this embodiment has an outer sleeve 251, which acts as a connecting sleeve for the guide 249 with a piston 253, and which is screwed by screws 255 to the guide 249, using the usual O-rings. The screws 255 can be used to adjust the axial pre-tensioning of the cage 217. The plunger 253 is connected by means of a screw thread 257 to a holder 259, which receives the toothed rod 207 at its distal end region.

There is a pronounced play Δ provided for between the guide 249 and the mount 259, which is bridged over via return springs 261, and which defines the pathway for the possible piston stroke.

When the piston 253 moves towards the cage, a stop 263 in the interior of the outer sleeve 251 provides a limit for the plunger stroke in this direction. To limit the piston stroke in the opposite direction, a pin is provided, which is inserted in the recess indicated by the reference 265, and which acts in combination with the outer circumference of the piston 253 by means of a corresponding ledge 267, in that the pin forms a stop for the ledge 267. This ensures that the guide 249 of the toothed rod 207 in the control element 209, which is connected by screwing the screw thread 257 to the piston 253, cannot be pulled right out from the outer sleeve 251. Sealing elements 271 ensure a proper closure of the interior of the control element 209 towards the outside.

At the distal termination of the guide 247 of the toothed rod 207 connected to the piston 253, and thus accessible from the outside, there is provided a slot-shaped recess 269, which serves as adjusting screw, to make it possible to modify the pre-tensioning of the cage manually there using a screwdriver.

Alternatively, in this embodiment it is also possible to connect a motor via this distal section of the control element 209 to the device 217, instead of the manual, post-operative adjustment by the patient himself or by the doctor attending to him.

The air column formed in the cage 217, which is at the usual air pressure as in the case of the other embodiments described previously, is thus exposed to the fluctuations in air pressure. Protracted series of tests have shown that in normal circumstances these air pressure fluctuations only cause a small change in the air column in the cage. Extreme or more extreme conditions, such as air pressure fluctuations when flying, or even at altitudes above at least 2000 m, may certainly cause a change.

Here additional provision has been made for combining the inventive device with sensors detecting movement, inclination and/or pressure. All three of the said types of sensor are already known in the field of medicine and are being used for various applications. It is advantageous for them to be fitted externally and thus avoid encumbering the implantation site unnecessarily.

The preliminary trials also covered a selective deployment of the said sensors as required. According to the preliminary tests it has been found that on airplane flights it suffices to just use a pressure sensor. In normal use it can be very comfortable to also use a motion sensor, which for example takes into account the rest-phases of the patient and prevents a re-adjustment from taking place during these times, by not passing on any signals to the pressure sensor. The coupling of the sensors together is possible for the specialist, according to present-day measurement and control technology, so it is not necessary to specially discuss this here.

Accordingly it is also possible to have a fully automatic re-adjustment. In fact this should even be the goal, so that it is not the patient, unfamiliar with the special field, who is operator of the device, but an automatic machine does the fine adjustment using the adjusting screw 269 or the motor. Regular inspections by the supervising doctor then complete the adjustment in a medically acceptable fashion.

1. A device for preventing incontinence, comprising: a tube-shaped body inside of which is a first longitudinal guide element; and a hose-like retaining element connected to the tube-shaped body and inside of which is a second longitudinal guide element that opens in the axial direction into a termination on the side thereof opposite the tube-shaped body, wherein the hose-like retaining element comprises a reversible stretchable and collapsible metal mesh having a polymer coating that is airtight and fluid-tight at the termination and in the region of the transition to the tube-shaped body.

2. The device according to claim 1; wherein the hose-shaped retaining element is filled with air at atmospheric pressure.

3.-20. (canceled)

21. The device according to claim 1; wherein the tube-shaped body comprises a tube-shaped, flexible hose made of at least one biologically compatible plastic or polymer, the material being selected from among polyurethanes, polyether block amides, polyamides, latex, polyvinyl chloride and/or silicone.

22. The device according to claim 21; wherein the tube-shaped, flexible hose is a fabric-reinforced and/or fabric-woven hose.

23. The device according to claim 21; wherein the tube-shaped, flexible hose has a metallic reinforcement.

24. The device according to claim 1; wherein the tube-shaped body comprises a tube-shaped shrink hose made of at least one biologically compatible thermoplastic selected from among polyolefins, polyvinyl chloride, polyvinylidene fluoride, polytetrafluorethylene and/or Viton, and the shrink hose and the hose-like retaining element with the metal mesh fit into each other.

25. The device according to claim 24; wherein the tube-shaped shrink hose has a metallic reinforcement, and the metallic reinforcement and the reversible stretchable and collapsible metal mesh of the hose-like retaining element are constructed as a sandwich between two plastic layers.

26. The device according to claim 1; wherein the tube-shaped body has an anti-microbial silver coating or a coating made of a diamond-like carbon.

27. The device according to claim 1; wherein the tube-shaped body has a generally closed enveloping sheath formed on its surface, and outward-facing fastening elements in the form of protrusions moulded on the tube-shaped body and constructed in one piece with the tube-shaped body.

28. The device according to claim 27; wherein the protrusions are moulded on the tube-shaped body at regular intervals in rows or at irregular intervals.

29. The device according to claim 27; wherein the protrusions are moulded on the tube-shaped body in the form of knobs, hooks and/or as scale-like protrusions.

30. The device according to claim 1; wherein the metal mesh is formed as a diamond pattern having rhombuses which are formed as slits when in the collapsed state and which extend in the lengthwise direction of the flexible hose.

31. The device according to claim 1; wherein the device is adjustable, and the tube-shaped body has as the first longitudinal guide element a flexible shaft approximately in the middle, which is held in the tube-shaped body by spacers.

32. The device according to claim 31; wherein the flexible shaft opens at its distal end section into a control element, which has a slot at its distal end for manual adjustment or for adjustment via connection to a motor as drive unit.

33. The device according to claim 1; wherein the tube-shaped body is formed as a tube at least partially surrounded by a sleeve, a pin is disposed inside the tube, the hose-like retaining element opens into an insert which receives the second longitudinal guide element in the form of a threaded rod, and the pin acts as a stop for the second longitudinal guide element and the insert.

34. The device according to claim 33; wherein the tube, the sleeve and the pin each have coupling elements which match up with an identical or complementary-fitting coupling element in the insert in the hose-like retaining element.

35. The device according to claim 1; wherein the device is actively adjustable, and the tube-shaped body has as the first longitudinal guide element a toothed rod, which is held in the tube-shaped body by spacers, and which can be moved via a lifting device mechanically by hand or motor-driven.

36. The device according to claim 35; wherein the actively adjustable device is coupled to a motion, inclination, pressure and/or volumetric sensor, and controlled by at least one of these sensors.

37. A device for preventing incontinence comprising two of the devices according to claim 1, which are each implantable adjacent to the urethra of a patient.

38. A device for preventing incontinence according to claim 37; wherein at least one of the devices is adjustable.
